(19) United States
(12) Patent Application Publication
(10) Pub. No.: US 2007/0248648 A1
(43) Pub. Date: Oct. 25, 2007

(54) High protein, fatigue reducing powder

(75) Inventors: Shawn Sherwood, Carlsbad, CA (US); David Jenkins, San Diego, CA (US)

Correspondence Address: Shirley L. Church, Esq.;DUCKOR SPRADLING METZGER & WYNNE, A Law Corporation, San Diego, CA, 92101-7909, US

(73) Assignee: Next Proteins, Inc.

(21) Appl. No.: 11/407,825

(22) Filed: Apr. 20, 2006

(51) Int. Cl. A61K 47/00; A61K 31/198

(52) U.S. Cl. 424/439; 514/563; 514/561

(57) ABSTRACT

A powdered composition which provides improved muscle growth and helps reduce fatigue, comprising a combination of edible nutritional whey protein, citrulline malate, and at least one branched chain amino acids. The branched chain amino acids are L-isoleucine, L-leucine, and L-valine. The concentration of nutritional protein present in the composition ranges from about 10% by weight to about 96% by weight, and may be about 20% by weight to about 90% by weight. The composition may contain additional ingredients such as polypeptides, a hyper-immune milk protein concentrate, amino acids, antioxidants and various flavorings and sweeteners. A method of use of the composition is also disclosed.

### RELATED APPLICATIONS

The present application is related to application Ser. No. 11/191,708, filed Jul. 27, 2005 entitled “Composition Permitting Muscle Growth While Protecting Joint Health” which is hereby incorporated by reference in its entirety.

### BACKGROUND OF THE INVENTION

1. Field of the Invention

The present invention pertains to a powdered high protein composition, and to a method of using the composition to improve muscle growth while reducing fatigue.

2. Brief Description of the Background Art

This section describes background subject matter related to the disclosed embodiments of the present invention. There is no intention, either express or implied, that the background art discussed in this section legally constitutes prior art.

Fatigue is an important factor that influences physical performance. It has two components, central fatigue and peripheral fatigue. Central fatigue has been described in the art as relating to neuronal inputs to muscle and the brain circuitry that drives them. Peripheral fatigue relates to nutritional, hormonal, and mechanical aspects of metabolism and physiology that directly affect muscle tissue. Dietary amino acids and protein have been examined for their affect on peripheral and central fatigue, particularly from the metabolic and neurochemical perspectives. Fernstrom and Wolfe, Introduction to symposium on branched-chain amino acids in exercise, J. of Nutrition 136: 524S (2006).

It is alleged in the art that (1) branched chain amino acids (BCAAs) can become depleted in muscle and plasma during exercise, producing a negative impact on muscle energy economy and promoting muscle fatigue, and (2) plasma BCAA depletion can indirectly increase tryptophan uptake into the brain, stimulate neuronal serotonin synthesis and release, causing central fatigue. Supplying BCAAs during training and competition has been described in the art as beneficial to both muscle function and mental focus by preventing BCAA depletion. Fernstrom, Branched-chain amino acids and brain function, J. of Nutrition 135: 1539S-1546S (2005) (please see page 1541S).

The BCAAs leucine, isoleucine, and valine account for 35-40% of the dietary essential amino acids in body protein and 14-18% of the total amino acids in muscle proteins. Shimomura et al., Nutraceutical effects of branched-chain amino acids on skeletal muscle, Journal of Nutrition 136: 529S-532S (2006) (please see page 529S).

BCAAs have been portrayed in the art as having anabolic effects on protein metabolism by increasing the rate of protein synthesis and decreasing the rate of protein degradation in resting human muscle. Also, during recovery from endurance exercise, BCAAs have been alleged to have anabolic effects in human muscle. Bloomstrand et al., Branched-chain amino acids activate key enzymes in protein synthesis after physical exercise, Journal of Nutrition 136: 269-273 (2006) (please see Abstract).

PCT Patent Publication Number WO 2004/049830, published Jun. 17, 2004 by applicant Tsuchita et al., describes an improver of muscular fatigue comprising 4 kinds of amino acids (leucine, isoleucine, valine and glutamine), and a whey protein component (whey protein and/or decomposition product of whey protein). At least one of a whey protein isolate (WPI), a whey protein concentrate (WPC), β-lactoglobulin, and α-lactalbumin is used as the whey protein.” (Please see the Abstract).

Citrulline malate is another anti-fatigue substance that has been described in the art. It has been suggested to have a protective effect against acidosis and ammonia poisoning, which may explain its anti-fatigue properties. Activity of citrullline malate on acid-base balance and blood ammonia and amino acid levels, Arzneimittelforschung 41: 660-663 (1991) (please see Abstract).

Citrulline malate is usually prescribed as an antiasthenic treatment. It is also known in the art by the trade name Stimol™ or the CAS registry number 5490-97-5, and it is a mixture of citrulline, which is involved in the urea cycle, and malate, a tricarboxylic acid cycle (TCA) intermediate. Bendahan et al., Citrulline/malate promotes aerobic energy production in human exercising muscle, British Journal of Sports Medicine 36: 282-289 (2002) (please see Introduction).

In a study by Bendahan et al., ingestion of 6 grams per day of citrulline malate for 15 days was alleged to result in a significant reduction in the sensation of fatigue. Citrulline/malate promotes aerobic energy production in human exercising muscle, British Journal of Sports Medicine 36: 282-289 (2002) (please see Abstract).

Japanese Patent Application Number JP1988000281672, published May 17, 1990 by Hiroko et al., describes an amino acid-containing food composition without the bitter taste stemmed from the amino acids. The composition described contains (A) amino acids(s) (pref. isoleucine, leucine, lysine, methionine, phenylalanine, tryptophan, valine, arginine, histidine, citrulline, omithine or proline) and/or peptide, and (B) tea and/or the flavoring component(s) thereof. The disclosure teaches that it is preferable that the amino acid concentration be 0.1 to 60 wt. % and the concentration of the tea and/or the flavoring component(s) thereof be 0.1 to 50wt. % in the final product. (Please see the Abstract).

Chinese Patent Application Number CN2003000117070, published Oct. 29, 2003 by applicants Qi and Jie, discloses a medical composition for treating various ailments, including chronic fatigue, which is prepared from ornithine and citrulline or their salts, jujube, ginkgo seed and ginseng. (Please see the Abstract).

U.S. Patent Publication No. 2005/0256192, published Nov. 17, 2005 by applicant Gardiner and Heuer describes a nutritional composition for enhancing lean muscle stimulus, growth, strength and recovery, supporting endurance, strength, performance, size and stamina in individuals. The nutritional composition may include L-arginine and creatine. In addition, the nutritional composition may also include L-citrulline, L-aspartic acid, ginseng root extract, and maritime pine (bark) extract or it may also include L-leucine and L-valine. (Please see the Abstract).

### DETAILED DESCRIPTION OF EXEMPLARY EMBODIMENTS

As a preface to the detailed description presented below, it should be noted that, as used in this specification and the appended claims, the singular forms “a”, “an”, and “the” include plural referents, unless the context clearly dictates otherwise.

In instances where the word “about” is used in this document, this indicates that the precision of a value is within about ±10%.

Disclosed herein is a powdered compound which can be mixed into various liquids and soft foods to provide improved muscle growth while simultaneously reducing fatigue. The powdered compound comprises a relatively high concentration of edible nutritional protein, citrulline malate, and branched chain amino acids (BCAAs). Despite the bitter and unpalatable taste of the citrulline malate and BCAAs individually, the powdered compound of the invention imparts an overall pleasing taste.

The nutritional protein is an edible nutritional whey protein. The concentration of whey protein present in the composition ranges from about 10% by weight to about 96% by weight, and may be about 20% by weight to about 90% by weight. The whey protein may be present as two ingredients, where a first ingredient is a whey protein isolate in which the whey protein concentration is at least 90% by weight. The second ingredient may be a whey protein concentrate in which the whey protein concentration ranges from about 25% by weight to about 89% by weight, and may be about 80% by weight.

The BCAAs include L-isoleucine, L-leucine, and L-valine. However, these BCAAs may be present individually or in various combinations. Each may be present at a concentration of about 0.01% by weight to 5.0% by weight. In one embodiment they are each present at a concentration of about 0.01% by weight to 0.50% by weight. In another embodiment they are each present at concentration of about 1.0% by weight to 2.0% by weight.

The citrulline malate ranges from about 1% by weight to about 15% by weight. In one embodiment the citrulline malate is present at about 10% by weight.

Polypeptides and amino acids of the kind which are related to muscle and nerve tissue development may be added optionally, for particular end use applications. Examples of polypeptides which may be added to the composition include glutamine peptides and hydrolyzed whey protein peptides having a molecular weight of less than about 10,000 Daltons (typically having a molecular weight ranging from about 1,000 to about 10,000 Daltons). Amino acids and their analogs and derivatives which may be added to the composition include phosphatidylcholine, Taurine, L-Glutamine, L-Arginine, L-Phenylalanine, L-Tyrosine, L-Citrulline, and N-acetylcysteine, by way of example and not by way of limitation. The concentration of amino acids and their analogs and derivatives in the composition ranges from about 0.01% by weight to about 15.0% by weight, more typically the concentration is in the range of about 0.11% by weight to about 11% by weight.

Antioxidants which provide antioxidant potentiating properties in the body may also be included in the composition. Examples of such antioxidants include lecithin, lactoperoxidase, and lactoferrin, not by way of limitation. Typically the concentration of antioxidants ranges from about 0.01% by weight to about 5.0% by weight in the composition, more typically the concentration is in the range of about 0.1 1% by weight to about 0.15% by weight.

A hyper-immune milk protein concentrate may be added to help protect joints. The concentration of this hyper-immune milk protein concentrate ranges about 1% by weight to about 20% by weight. In one embodiment the hyper-immune milk protein is present at about 7% by weight.

A preservative of the kind known in the food industry may be added to provide freshness. A preferred preservative is tocopherol (vitamin E). The concentration of preservative ranges from about 0.01% by weight to about 1.0% by weight. In one embodiment the concentration of preservative is in the range of about 0.01% by weight to about 0.015% by weight.

Various flavorings and sweeteners (preferably artificial sweeteners to reduce the amount of carbohydrates in the compound) may be added to provide palatability. For example, vanilla flavorings may be present at a concentration ranging from about 1% by weight to about 5% by weight. Artificial sweeteners may be present at a concentration ranging from about 0.1% by weight to about 1.0% by weight. In one embodiment artificial sweetener is present at about 0.11% by weight.

One skilled in the art of food and dietary supplements will recognize that there are a number of different flavorings and antioxidants which may be used in combination with the edible nutritional whey protein, branched chain amino acids, and citrulline malate. In addition, there are a variety of polypeptides and amino acids which are known to be beneficial to the body and which may be added to the composition as well, for particular remedial health benefits. It is not intended that the scope of the invention be limited to the exemplary compositions described herein.

A recommended serving of the composition ranges from 1 gram to 100 grams, depending on consumer goals. In one regimen of use, a consumer, upon initiating use of the compound, begins by using two to three 30 gram servings per day, one of which is typically in the morning or before exercising.

### EXAMPLES

### Example One

### Composition Ingredients and Method of Making the Composition

| INGREDIENTS | PERCENTAGE BY WEIGHT |
|---|---|
| Whey Protein | 85 |
| Non-nutritive Sweeteners | 0.1 |
| Flavoring | 0.9 |
| L-Isoleucine | 1 |
| L-Valine | 1 |
| L-Leucine | 2 |
| Citrulline Malate | 10 |

*The above-listed ingredients are agglomerated using a wetting and drying process in a fluid bed agglomerator of the kind commonly used in the industry.

The agglomerated composition is easily mixed into water using a spoon, where there is immediate dispersion and rapid dissolution of the compound. This composition may be mixed into other liquids or soft food which already have a distinctive flavor.

The composition described above is particularly palatable. This pleasant taste of the overall composition is an unexpected result, because the composition contains the bitter and unpleasant tasting branched chain amino acids and citrulline malate.

The composition described above is available in multiple flavors, including, but not limited to orange, fruit punch, grape, lemon, lime, tropical fruit, vanilla, strawberry, and chocolate. This product will be marketed for use anytime the consumer chooses to consume it.

### Example Two

### Composition Ingredients and Method of Making the Composition

| INGREDIENTS | PERCENTAGE BY WEIGHT |
|---|---|
| Whey Protein | 27 |
| Soy Lecithin | 0.5 |
| Vitamin E | 0.01 |
| Caffeine | 1.03 |
| Non-nutritive Sweeteners | 0.55 |
| Nutritive Sweeteners | 26.3 |
| Citric Acid | 6.75 |
| Ascorbic Acid | 1.0 |
| Malic Acid | 1.0 |
| Flavoring | 2.67 |
| L-Phenylalanine | 0.20 |
| Taurine | 3.35 |
| L-Tyrosine | 10.2 |
| Magnesium Oxide | 1.95 |
| L-Citrulline | 0.10 |
| Pantothenic Acid | 0.01 |
| DMAE Bitartrate | 1.0 |
| Glucuronolactone | 2.0 |
| Green Tea | 0.20 |
| Vinpocetine | 0.015 |
| Vitamin B12 | 0.002 |
| Chromium Aspartate | 0.04 |
| Niacinimide | 0.04 |
| L-Isoleucine | 0.01 |
| L-Valine | 0.01 |
| L-Leucine | 0.50 |
| Citrulline Malate | 10.0 |
| Pyridoxine HCL | 0.10 |
| Magnesium Orotate | 1.0 |
| Betaine Annhydrous | 0.50 |
| Magnesium Aspartate | 0.20 |

*The above-listed ingredients are agglomerated using a wetting and drying process in a fluid bed agglomerator of the kind commonly used in the industry.

The agglomerated composition is easily mixed into water using a spoon, where there is immediate dispersion and rapid dissolution of the compound.

The composition described above is particularly palatable. This pleasant taste of the overall composition is an unexpected result, because the composition contains the bitter and unpleasant tasting branched chain amino acids and citrulline malate.

The composition described above is available in multiple flavors, including, but not limited to orange, fruit punch, grape, lemon, lime, tropical fruit, vanilla, strawberry, and chocolate. This product is particularly useful for use prior to and during a workout. One embodiment of the composition (described above) has been designated “Ultimate Orange” for marketing purposes.

### Example Three

### Composition Ingredients and Method of Making the Composition

| INGREDIENTS | PERCENTAGE BY WEIGHT |
|---|---|
| Whey Protein | 72.4 |
| Hyper-Immune Milk Protein Concentrate | 7 |
| Sucrose | 2 |
| Flavoring | 1.5 |
| L-Isoleucine | 1.5 |
| L-Valine | 1.5 |
| L-Leucine | 2 |
| Citrulline Malate | 10 |
| Calcium Carbonate | 2 |
| Sucralose | 0.05 |
| Acesulfame Potassium | 0.05 |

*The above-listed ingredients are agglomerated using a wetting and drying process in a fluid bed agglomerator of the kind commonly used in the industry.

The agglomerated composition is easily mixed into water using a spoon, where there is immediate dispersion and rapid dissolution of the compound. This composition may be mixed into other liquids or soft food.

The composition described above is particularly palatable. This pleasant taste of the overall composition is an unexpected result, because the composition contains the bitter and unpleasant tasting branched chain amino acids and citrulline malate.

The composition described above is available in multiple flavors, including, but not limited to orange, fruit punch, grape, lemon, lime, tropical fruit, vanilla, strawberry, and chocolate. This product contains the joint protecting hyper-immune milk protein concentrate. It will be marketed for use anytime the consumer chooses to consume it.

### Example Four

### How to Prepare the Composition

There are a number of ways to prepare the composition for consumption. It may be mixed with a spoon, shaker, or may be blended into a delicious shake with the consumer's favorite ingredients. For example, one serving (about 30 grams, which may be scooped from a container of the composition in granular form) may be added to a blender filled with 3-6 oz. of the consumer's favorite beverage. The blender cover is applied, and the mixture is blended for 20-30 seconds. For additional enjoyment, 1-2 ice cubes may be added, followed by additional blending for a time period of about 30 seconds. Another approach is to fill a shaker bottle with 3-6 oz. of the consumer's favorite beverage, add one serving of the composition, cover and shake for 20-30 seconds. An additional method of preparation is to add one serving to a tall glass, add 3-6 oz. of the consumer's favorite beverage, mix with a spoon for about 20 seconds or until the composition is well dispersed.

### Example Five

### How to Use the Composition

One embodiment of the present invention is a method of increasing muscle growth, while simultaneously reducing fatigue by consuming daily a combination of ingredients, wherein the combination includes (a) whey protein in an amount ranging from about 1 gram to about 250 grams, (b) citrulline malate in an amount ranging from about 0.01 grams to about 10 grams, (c) L-leucine in an amount ranging from about 0.01 grams to about 21 grams, (d) L-isoleucine in an amount ranging from about 0.01 grams to about 21 grams, (e) L-valine in an amount ranging from about 0.01 grams to about 21 grams, and (f) hyper-immune milk protein in an amount ranging from about 0.5 grams to about 6 grams. This combination of ingredients may be consumed in one serving or a plurality of servings, for example this combination of ingredients may be consumed in 2 daily servings. The combination of ingredients may be mixed with consumer's favorite ingredients, as describe above in Example 4.

Another embodiment of the present invention is a method of increasing muscle growth, while simultaneously reducing fatigue by consuming daily a combination of ingredients, wherein the combination includes (a) whey protein in an amount ranging from about 50 gram to about 100 grams, (b) citrulline malate in an amount ranging from about 5 grams to about 10 grams, (c) L-leucine in an amount ranging from about 5 grams to about 15 grams, (d) L-isoleucine in an amount ranging from about 5 grams to about 15 grams, (e) L-valine in an amount ranging from about 5 grams to about 15 grams, and (f) hyper-immune milk protein in an amount ranging from about 2 grams to about 5 grams. This combination of ingredients may be consumed in one serving or a plurality of servings, for example this combination of ingredients may be consumed in 2 daily servings. The combination of ingredients may be mixed with consumer's favorite ingredients, as describe above in Example 4.

An alternative embodiment of the present invention is a method of increasing muscle growth, while simultaneously reducing fatigue by consuming daily a combination of ingredients, wherein the combination includes (a) about 60 grams of whey protein, (b) about 6 grams of citrulline malate, (c) about 14 grams of L-leucine, (d) about 14 grams of L-isoleucine, (e) about 14 grams of L-valine, and (f) about 4 grams of hyper-immune milk protein. This combination of ingredients may be consumed in one serving or a plurality of servings, for example this combination of ingredients may be consumed in 2 daily servings. The combination of ingredients may be mixed with consumer's favorite ingredients, as describe above in Example 4.

While the invention has been described in detail above with reference to several embodiments, these embodiments are not intended to limit the scope of the invention. Various modifications within the scope and spirit of the invention will be apparent to those familiar with the industry of health care foods and supplements, who can expand the embodiments to correspond with the subject matter of the invention claimed below.

We claim:

1. A powdered composition which is used to improve muscle growth and reduce fatigue, wherein the composition comprises:
(a) an edible nutritional whey protein, wherein the whey protein is present in the composition at a concentration ranging from about 10% by weight to about 96% by weight;
(b) citrulline malate, wherein the citrulline malate is present in the composition at a concentration ranging from about 1% by weight to about 15% by weight; and
(c) branched chain amino acids, wherein the branched chain amino acids are selected from the group consisting of L-isoleucine, L-leucine, and L-valine, and combinations thereof, where each branched chain amino acid which is present is at a concentration of about 0.01% by weight to about 5.0% by weight.

2. A composition in accordance with claim 1, wherein the whey protein is present in the composition at a concentration ranging from about 20% by weight to about 90% by weight.

3. A composition in accordance with claim 1, wherein the citrulline malate is present in the composition at a concentration of about 10% by weight.

4. A composition in accordance with claim 1, wherein each branched chain amino acid which is present is at a concentration of about 0.01% by weight to about 0.50% by weight.

5. A composition in accordance with claim 1, wherein each branched chain amino acid which is present is at a concentration of about 1.0% by weight to about 2.0% by weight.

6. A composition in accordance with claim 1 or 2, wherein a hyper-immune milk protein concentrate is present.

7. A composition in accordance with claim 1 or claim 2, wherein polypeptides and amino acids of the kind which are beneficially related to muscle and nerve tissue development are present.

8. A composition in accordance with claim 1 or claim 2, wherein antioxidants are present.

9. A composition in accordance with claim 1 or claim 2, wherein flavorings are present.

10. A composition in accordance with claim 1 or claim 2, wherein sweeteners are present.

11. A method of increasing muscle growth while simultaneously reducing fatigue by consuming daily a combination of ingredients, wherein the combination includes (a) whey protein in an amount ranging from about 1 gram to about 250 grams, (b) citrulline malate in an amount ranging from about 0.01 grams to about 10 grams, (c) L-leucine in an amount ranging from about 0.01 grams to about 21 grams, (d) L-isoleucine in an amount ranging from about 0.01 grams to about 21 grams, (e) L-valine in an amount ranging from about 0.01 grams to about 21 grams, and (f) hyper-immune milk protein in an amount ranging from about 0.5 grams to about 6 grams.

12. A method in accordance with claim 11, wherein the combination of ingredients is consumed in a plurality of servings.

13. A method in accordance with claim 12, wherein the combination of ingredients is consumed in two servings.

14. A method in accordance with claim 11, wherein the combination of ingredients includes (a) whey protein in an amount ranging from about 50 gram to about 100 grams, (b) citrulline malate in an amount ranging from about 5 grams to about 10 grams, (c) L-leucine in an amount ranging from about 5 grams to about 15 grams, (d) L-isoleucine in an amount ranging from about 5 grams to about 15 grams, (e) L-valine in an amount ranging from about 5 grams to about 15 grams, and (f) hyper-immune milk protein in an amount ranging from about 2 grams to about 5 grams.

15. A method in accordance with claim 14, wherein the combination of ingredients is consumed in a plurality of servings.

16. A method in accordance with claim 15, wherein the combination of ingredients is consumed in two servings.

17. A method in accordance with claim 11, wherein the combination of ingredients includes (a) about 60 grams of whey protein, (b) about 6 grams of citrulline malate, (c) about 14 grams of L-leucine, (d) about 14 grams of L-isoleucine, (e) about 14 grams of L-valine, and (f) about 4 grams of hyper-immune milk protein.

18. A method in accordance with claim 17, wherein the combination of ingredients is consumed in a plurality of servings.

19. A method in accordance with claim 18, wherein the combination of ingredients is consumed in two servings.
